(19) United States
(12) Patent Application Publication
(10) Pub. No.: US 2025/0114540 A1
(43) Pub. Date: Apr. 10, 2025

(54) HANDHELD SKIN TREATMENT DEVICES

(71) Applicant: AGE SCIENCES, INC. dba PMD BEAUTY, Draper, UT (US)

(72) Inventors: Sam ALEXANDER, Alpine, UT (US); Wu Jian HUA, Shenzhen (CN); Benjamin Ryan MOORE, Dongguan (CN); James Grant GHORMLEY, IV, Arlington, TX (US)

(21) Appl. No.: 18/729,945

(22) PCT Filed: Feb. 14, 2023
(86) PCT No.: PCT/US23/13003

§ 371 Date: Jul. 18, 2024

Related U.S. Application Data
(60) Provisional application No. 63/309,811, filed on Feb. 14, 2022.

(51) Int. Cl. A61M 11/00

(52) U.S. Cl. CPC A61M 11/005; A61M 11/001; A61M 2205/0211; A61M 2210/04; A61M 2210/0606

(57) ABSTRACT

A handheld skin treatment device can include an atomizer to generate water droplets when fed from a water reservoir, a water supply tank to contain the water reservoir and to feed water therefrom to interact with the atomizer and generate the water droplets, and a heating tube to receive and heat the water droplets having an average temperature from 55° C. to 90° C. at an exit opening of the heating tube.

### BACKGROUND

There are many different types of skin treatment devices. Some devices are referred to generally as steamers, or face steamers. These types of devices may generate steam using heat to be directed toward or applied to the face, for example. Other “steamers” do not generate steam per se, but rather generate fine water droplets that can be applied as a heated mist to the skin. These types of treatments may benefit the skin when applied therapeutically. Some benefits may vary, depending on the specific technology, but may include moisturizing and/or hydrating of the skin, oil control, makeup fixing and/or removal, reduction of fine wrinkles, tightening of the skin, smoothing of the skin, reduction in skin sensitivity, reduction in redness and/or pain, unclogging or dredging pores (such as for cleansing skin, improved natural feel and skin appearance with or without makeup, and/or allowing better penetration of follow-up product application), detoxifying skin, enhancing water penetration, speeding up blood circulation, eliminating dirt, reducing dark spots, enhancing the healthy appearance of skin, skin and/or hair (including eyelash) nourishment, etc.

Most of the small face steamers on the market mostly have a placement structure that uses a horizontal sprayer, with a sprayer opening at or beneath the location where water is fed through the sprayer tube/nozzle. Furthermore, the delivery tube, heating stroke, and output pattern of useable hot water droplets tend to be small. For example, with the use of short tubes, relative low wattage of power delivered to the heating tube, and the horizontal delivery configuration, the heating 30 power delivered to the water droplets tends to be low, meaning that the delivery of heated mist to the skin occurs in very close proximity to the skin, i.e. which reduces the pattern size of water droplet delivery. As an example, at 5 cm from the delivery end of the heating tube, with many systems, the heated mist cools to close to room temperature, reducing the benefit of the added heat to the droplets. On the other hand, units that deliver higher temperature mist tend to not be handheld, and thus, can be cumbersome to use and transport.

### BRIEF DESCRIPTION OF THE DRAWINGS

FIG. 1A is a perspective view of the outer shell of an example handheld skin treatment device in accordance with the present disclosure;

FIG. 1B is a cross-sectional view of an example handheld skin treatment device in accordance with the present disclosure;

FIG. 2 is a partially exploded perspective view of an example handheld skin treatment device in accordance with the present disclosure;

FIG. 3 is a perspective view of a heating tube electrically connected to a control circuit board of an example handheld skin treatment device in accordance with the present disclosure;

FIG. 4 is a schematic diagram of an example heated droplet generator of an example handheld skin treatment device in accordance with the present disclosure;

FIG. 5 is an exploded view of a heated droplet generator of an example handheld skin treatment device in accordance with the present disclosure; and

FIG. 6 is a schematic diagram of an example fixing adapter of an example heated droplet generator in accordance with the present disclosure.

### DETAILED DESCRIPTION

The present disclosure is drawn to handheld skin treatment devices, and more particularly, handheld skin treatment devices that can deliver heated atomized water for application to any skin surface, including facial skin at a high, safe temperature with a relatively large delivery pattern in some examples.

In one example, a handheld skin treatment device can include an atomizer to generate water droplets when fed from a water reservoir, a water supply tank to contain the water reservoir and to feed water therefrom to interact with the atomizer and generate the water droplets, and a heating tube to receive the water droplets and to generate heated water droplets having an average temperature from 55° C. to 90° C. at an exit opening of the heating tube.

In another example, a method of treating a skin surface with a handheld device can include atomizing water fed from a water reservoir held by a water supply tank within the handheld device to form water droplets, passing the water droplets through a heating tube to form heated water droplets having an average temperature from 55° C. to 90° C. at an exit opening of the heating tube, and directing the heated water droplets from the exit opening toward a skin surface, wherein the exit opening is positioned at from 5 cm to 15 cm from the skin surface to provide delivery of the heated water droplets at a cooled temperature from about 35° C. to about 45° C. In some examples, the skin surface can be a facial skin surface. In other examples, the delivery of the heated water droplets at the cooled temperature can provide a delivery pattern having a skin surface contact area at least 10 times larger in area than the area of the exit opening, e.g., from 10 times to 25 times larger in area than the area of the exit opening. In other examples, the heating tube can be capable of delivering the heated water droplets from the exit opening while the heating tube is in any orientation. In still other examples, the method can include reducing a volume of the water droplets that collect at the atomizer used for atomizing water by vaporizing water droplets collected on an interior surface of the heating tube, absorbing runoff of water droplets along the interior surface of the heating tube at an absorption assembly, or both.

In accordance with this, handheld skin treatment devices, methods of treating a skin surface with a handheld skin treatment device, and other related devices, systems, and methods described herein are described independently herein to some extent, but it is understood that discussions of any example herein can be considered applicable to all other examples, whether or not they are explicitly discussed in the context of that example. Thus, for example, when discussing a heating tubing in the context of the handheld skin treatment device, such disclosure is relevant to and directly supported in the context of the related systems and/or methods, and vice versa. Furthermore, it is noted that terms used herein will have the ordinary meaning in their technical field unless specified otherwise. In some instances, there are terms defined more specifically throughout the specification, and thus, these terms can have a meaning as described herein.

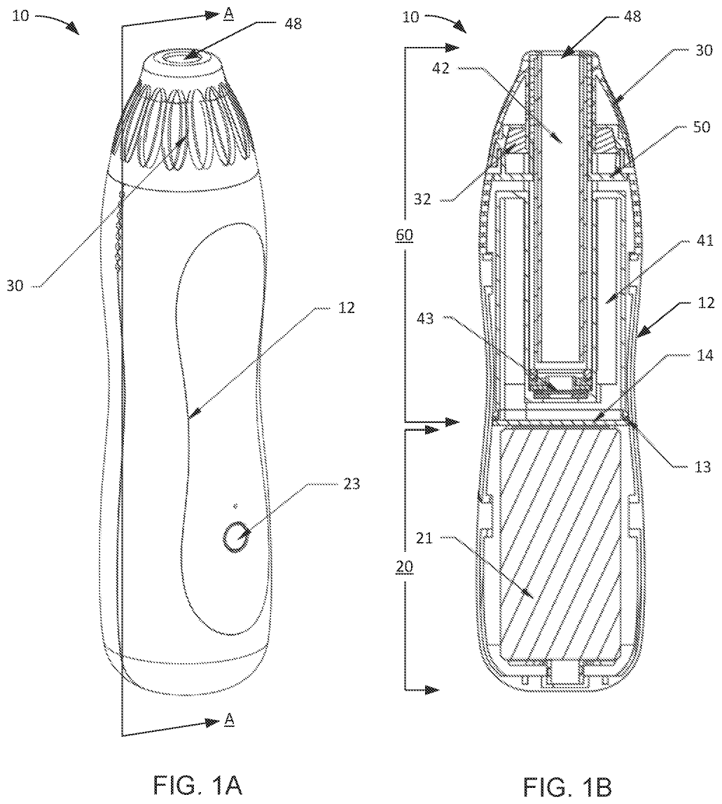

Referring now to FIGS. 1A and 1B, a handheld skin treatment device 10 is shown as a perspective view (FIG. 1A) and in cross-section (FIG. 1B) taken at A-A of FIG. 1A. The handheld skin treatment device in this example can include an atomizer 43 to generate water droplets when fed from a water reservoir, a water supply tank 41 to contain the water reservoir and to feed water therefrom to interact with the atomizer and to generate the water droplets, and a heating tube 42 to receive the water droplets and to generate heated water droplets having an average temperature from 55° C. to 90° C. at an exit opening 48 of the heating tube. Also shown in this example is a handheld housing 12 that is connected to a sprayer head 30, a sealing ring 13 and a partition 14 to separate a wet portion 60 from an electrical portion 20 of the handheld skin treatment device. The electrical portion, as shown, includes a control button 23 and a battery 21 to operate and power the handheld skin treatment device, among other components described hereinafter. The wet portion further includes a fixing adapter 50 to stabilize the heating tube and provide an opening for refilling the water supply tank, and a water retainer 32, e.g., silicone or other rubber retaining ring, and seal openings of the fixing adapter that are used to refill the water supply tank.

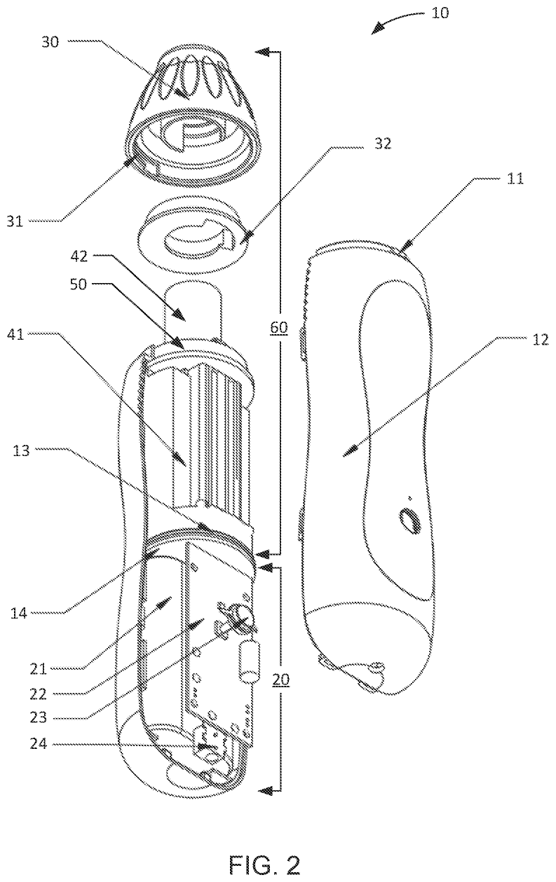

A partially exploded perspective view of an example handheld skin treatment device 10 is shown in FIG. 2, which provides additional detail that is more visibly apparent in this partially exploded perspective view. In this example, some of the same features as shown in FIGS. 1A and 1B are shown, such as the handheld housing 12 which houses the electrical portion 20 and the wet portion 60 of the device, separated by the partition 14 and the sealing ring 13 which seals the electrical portion from the wet portion of the handheld skin treatment device within the handheld housing.

In this example, the electrical portion 20 of the handheld skin treatment device includes a battery 21 and the control button 23, as previously described, but can also include a control circuit board 22, e.g., printed circuit board assembly (PCBA) or other circuit board for controlling operation, and a power interface 24 to interact with an electrical charger and/or power cord. The electrical charger can be a charging cord or may include a charging stand, for example (not shown). In one specific example, the battery, the control button, and the power interface can be electrically connected to a PCBA, which are carried within the housing within the electrical portion of the device. The housing may include openings for the electrical charger (not shown) and the control button to pass through for normal operation. In some examples, the PCBA can include a timer for automatic shutoff at a time frame set by the user and/or by the manufacturer. For example, a 5 minute to 12 minute treatment time can be set or selected by the user, e.g., the timer shuts off the heater and atomizer at about 8 minutes. There may also be a sensor associated with the water reservoir, and the PCBA can include shutoff circuitry when the water supply tank 41 is near empty or empty.

The wet portion 60 of the handheld skin treatment device 10 in this view also shows several of the features shown in FIGS. 1A and 1B in some additional detail. The handheld housing 12 can carry a sprayer head 30 attachable thereto via a housing tightener 11. For example, a sprayer head tightener 31 can be present on the sprayer head that inversely matches a housing tightener for securing the sprayer head to the housing. A fixing adapter 50 and a water retainer 32 provide an opening(s) and sealing of the opening(s), respectively, for refill access and closure of a water supply tank 41. A heating tube 42 can then heat atomized water supplied from the water supply tank and atomizer (not shown in this view) as it passes therethrough and out of the exit opening 48 for user interaction with the heated water droplets. The heating tube is shown as being partially supported by a fixing adapter 50, which also provides openings for filling the water supply tank and attachment mechanisms for interaction with the handheld housing as well as the sprayer head via the water retainer, for example.

Notably, the term “handheld” or “handheld housing” refers to a device or housing that contains the components described for operation, without the need of an external appendage or device connected thereto for operation. Thus, in one example, all of the components used to operate the handheld skin treatment device are contained within the handheld housing, including the water supply tank 41, the power supply, e.g., battery, circuitry, etc. Thus, when in use, the operator may operate the device without the use of a connected water supply. Thus, the water supply can be contained within the handheld device and no external water source is needed for operation for the duration of a single full treatment regimen, e.g., from 1 minute to 20 minutes, from 2 minutes to 15 minutes, or from 3 minutes to 10 minutes. A connected electrical power cable or charging source may remain connected, or the handheld skin treatment device may operate on battery power (either disposable battery power or rechargeable battery power), for example.

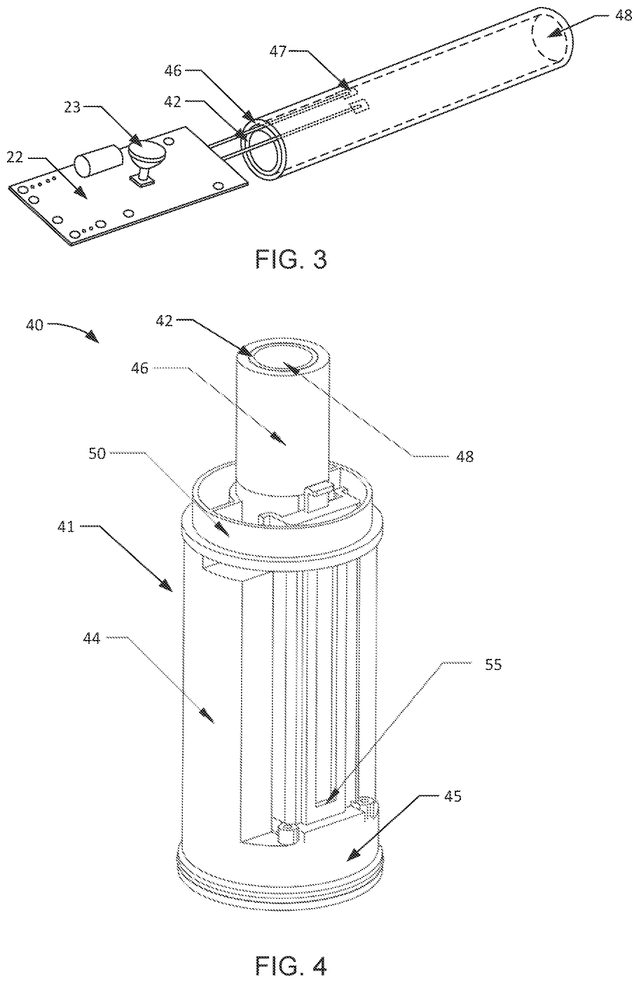

The electrical connection between the heating tube 42 and the control circuit board 22 is shown in FIG. 3. Both of these components can be installed within the handheld housing (not shown in this FIG.). The control circuit board includes various control circuitry and a control button 23 which can be used to turn on the handheld skin treatment device, as shown. For example, a simple single button (or multiple buttons or controls) can send power to the atomizer and the ceramic tube for heating. In this arrangement with relative high wattage used to operate the heater, in most instances, the majority of the power will typically go to the heating tube and a smaller amount of power will go to the atomizer. In some examples, the control button can also be used to turn off the device and/or to activate different settings. e.g., multiple temperature settings, multiple time of operation settings, multiple water atomization outputs, etc. For example, depressing the control button multiple times and/or for longer periods of time can be used to indicate different settings and/or on/off modes. There could alternatively be multiple buttons with different effects or modes and/or other control mechanisms in addition to or as an alternative to the control button, e.g., switches, dials, gesture control, orientation awareness, Bluetooth (or other wireless) connection for control from a smart device, etc.

The control circuit board 22 is shown as being electrically connected to the heating tube 42 by an electrical heater 47. The electrical heater may include, for example, electrical leads, resistive heaters, contact pads or strips, and/or any other components that can be used to transfer heat, via electrical input, to the bulk of the heating tube. In some examples, the electrical heater may include a separate heating component in thermal contact with the heating tube, and in other examples, the electrical heater can leverage the material of the heating tube to provide heat to the bulk of the heating tube. In some examples, the heating tube can be constructed of metal, such as elemental or metal alloy, ceramic material, a composite of metal and ceramic, and/or any other heat conducting or semi-conducting material that can transfer sufficient heat to the inside of the heating tube to cause the water droplets to become heated at the exit opening 48. In one example, the heating tube may be a metal heating tube with an interior surface coated or composited with a ceramic material.

Because the heating tube 42 can get very hot, e.g., a temperature that may burn the skin if contacted and/or damage adjacent components in some instances (depending on the materials used), the heating tube is sheathed within a thermal insulator 46. The thermal insulator can also be positioned around the heating tube to retain heat at the heating tube, e.g., to reduce the degree of heat dissipation, thus concentrating the heat at the heating tube for enhanced efficiency. The thermal insulator can be of any material or thickness that can protect the user from unwanted contact and assist with heat concentration at the heating tube, such as a heat insulating silica gel, aerogel, ceramic, polyurethanes, mineral wool, etc.

In further detail regarding the heating tube 42 and operation of the control circuit board 22, as mentioned, the operation may use alternating current or direct current (battery power). In either case, the power source may provide from 40 Watts to 100 Watts or from 50 Watts to 90 Watts of power to the heating tube. Not shown in this FIG. is the atomizer that is used to generate water droplets from a water reservoir upon delivery to the heating tube, but the atomizer can generate water droplets from the water reservoir supply at a particle size from 2 μm to 15 μm for heating. This relatively high wattage can provide a heating tube that generates heated water droplets (after atomization) having an average temperature from 55° C. to 90° C. at an exit opening 48 of the heating tube. Other temperature ranges of the heated water droplets may be from 60° C. to 85° C., or from 65° C. to 80° C., for example. Tube length may be sufficient to generate this level of heating, but should not be so long that too high of a volume of water droplets collect on the inner side walls of the heating tube. In some examples, the tube length may be from 5 cm to 20 cm, from 5 cm to 15 cm, or from 6 cm to 12 cm. Tube diameter (on the inside or open space) may be from 0.75 cm to 3 cm, from 0.9 cm to 2 cm, or from 1 cm to 1.5 cm, for example. With this configuration, or others, a user can position the exit opening at a sufficient distance from the skin to generate a larger pattern of heated water droplets for delivery than with devices of lower power levels. For example, water droplets generated by the atomizer and delivered to the heating tube can be heated sufficiently so that delivery of the heated water droplets to the skin surface may range from 35° C. to 45° C., from 37° C. to 43° C., or from 40° C. to 45° C. when the exit opening is positioned from 5 cm to 15 cm, from 5 cm to 12 cm, from 7 cm to 12 cm, or from 5 cm to 10 cm from the skin surface. As a specific example, water droplets having an average droplet size from 6 μm to 8 μm may be heated to about 70° C. at the exit opening and provide 40° C. to 45° C. user experience with the exit opening positioned about 5 cm to 10 cm from the skin surface.

In order to provide a high temperature to the water droplets, e.g., up to 90° C. at the exit opening 48, the heating tube 42 can be heated to a temperature so that the inner surface of the heating tube may be above the vaporization temperature of the water droplets. Thus, as water that does not exit the heating tube as water droplets collects on the inner side walls of the heating tube, the temperature may be, in some examples, sufficient to vaporize the water droplets. By vaporizing water droplets that coalesce on an inner surface of the heating tube, less water is permitted to run down the tube and interfere with the atomizer (not shown). Thus, by using higher temperatures for the heating tube with higher volumes of water atomization, greater water delivery to the skin can occur over a larger area of the skin (due to additional distance between the exit opening and the skin to allow for some droplet cooling to occur), and furthermore, this higher temperature has the added benefit of burning off some water condensation that inevitably occurs, prolonging the operable time the atomizer can be operational.

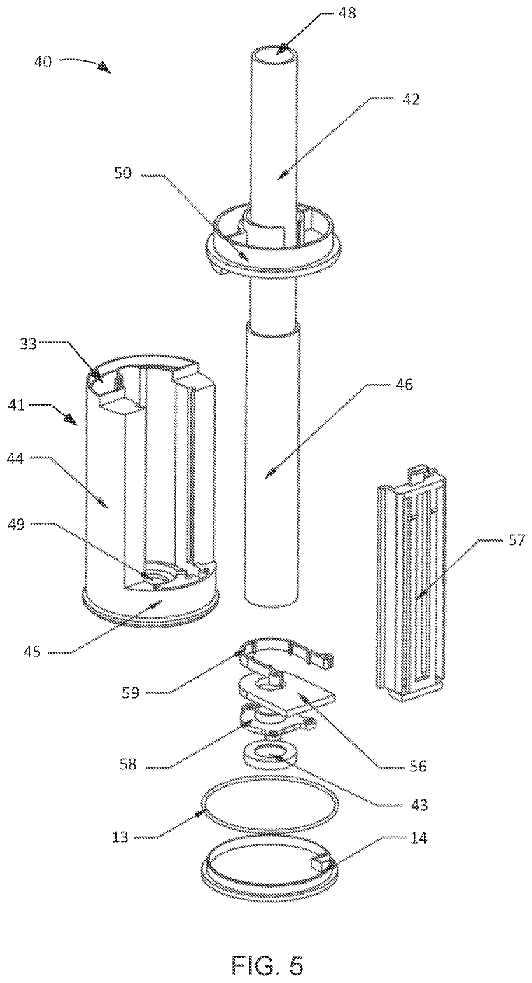

The heated droplet generator 40 of the handheld skin treatment device is shown as assembled in FIG. 4. The heated droplet generator is installed as part of the wet portion of the handheld skin treatment device within the handheld housing as previously shown. In this FIG., for additional clarity of this view, the housing, the sprayer head, and the water retainer are not shown. Here, a heating tube 42 and an exit opening 48 thereof is shown with a thermal insulator 46 surrounding the heating tube. The heating tube (and thermal insulator) is partially surrounded by a water supply tank 41. The water supply tank includes a lower tank portion 45 and an upper tank portion 44. The terms “upper” and “lower” are relative terms when in the orientation shown, such as may be the orientation when filling the water supply tank in this example, but it is understood that in operation, during charging, and/or during storage, the device may be positioned in any orientation. Thus, water can be filled in the water supply tank at the top of the upper tank portion, and water can be delivered to an atomizer (not shown) from the lower tank portion. The ports for water fill and delivery to the heating tube are shown in greater detail in FIG. 5.

Though the handheld skin treatment device can be used in any orientation, longer operation times may be achievable when the heating tube is orientated from horizontal to about 60° (with the exit opening positioned at a higher elevation than the atomizer), from horizontal to about 45°, from 5° to about 60°, or from 5° to about 45°. This allows for acceptable feed of the water from the water reservoir to the atomizer while reducing water runoff along the interior walls of the heating tube that collects at the atomizer. In more vertical orientations, e.g., from greater than about 60° to 90° (vertical), operation is also possible for shorter durations of time. This provides the ability of the user to treat the skin while changing the orientation of the handheld skin treatment device during use to vertical for short periods of time as may be convenient, e.g., the handheld skin treatment device may be oriented near vertically or vertically while treating the front of the face or cheeks and then oriented near vertically or vertically while treating the skin beneath the chin without ceasing operation.

On the opposite side of the heating tube 42 and thermal insulator 46 relative to the water supply tank 41 is an absorption assembly 55. The absorption assembly can be used to wick atomized water droplets away from the atomizer (not shown, but shown in FIG. 5) when the atomized water droplets contact and run down inner side walls of the heating tube (away from the exit opening 48). As the atomizer gets too wet, it can cease to operate properly. Thus, the absorption assembly can prolong the amount of time that the handheld skin treatment device can be operable as expected. The absorption assembly can be positioned so that it can partially or fully dry between uses and/or be periodically removed for dying and/or replacement.

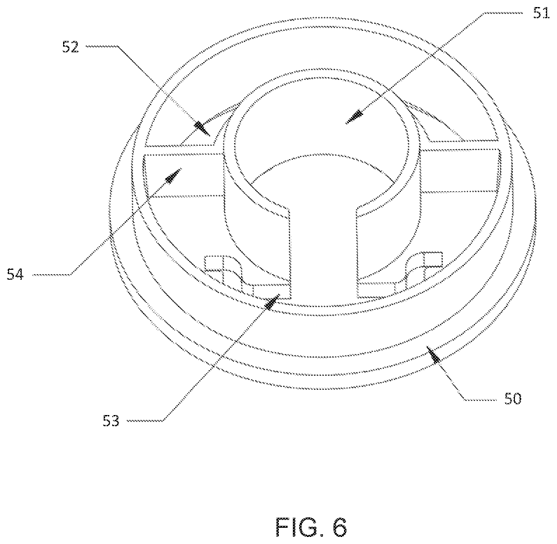

In further detail, FIG. 4 also depicts a fixing adapter 50 in place that provides a mechanism for additional stabilization of the heating tube 42, openings for filling the water supply tank, and attachment mechanisms for interaction with the handheld housing, sprayer head, and water retainer (not shown). Additional detail related to the fixing adapter is shown by way of example in FIG. 6 hereinafter. Also, it is noted that in the arrangement shown, the heating tube is configured to pass through an opening defined by an outer wall of the water supply tank 41, i.e. the water supply tank at the upper tank portion 44 laterally partially surrounds the heating tube with a C-shaped horizontal cross-section. Furthermore, the lower tank portion 45 is where the water is delivered to the heating tube from one end of the heating tube and the exit opening 48 is positioned beyond (above in this orientation) the upper tank portion of the water supply tank. This allows for a compact design that permits a wand-shaped handheld skin treatment device, rather than a more bulky configuration with a water supply that delivers water droplets in a direction perpendicular to the bulk of the water reservoir.

FIG. 5 depicts an exploded view of many of the components of the heated droplet generator 40, which is part of the wet portion of the handheld skin treatment device. The wet portion is separated from the electrical portion (not shown) by a partition 14 and a sealing ring 13 to prevent water from escaping from the bottom of the wet portion either exterior of the handheld housing and/or into the electrical portion. The water supply tank is as described in FIG. 4, with an upper tank portion 44 and a lower tank portion 45. However, in this FIG., at the top of the upper tank portion is shown a filling port 33, which is where water is added to the upper tank portion. Furthermore, a water delivery port 49 is shown where the water from the lower tank portion is delivered therethrough to the heating tube as atomized water droplets for heating. The atomized droplets are formed by passing water from the water supply tank through the water delivery port and into the heating tube. As shown in this example, the heating tube is sheathed by a thermal insulator, and may be supported and/or connected to other components using a fixing adapter 50, which is described in greater detail in FIG. 6 hereinafter.

Positioned between or otherwise at or near the interface between the water delivery port 49 and the heating tube 42 is an atomizer 43, which may be an ultrasonic atomizer, a piezoelectric atomizer, a nebulizer, or other device that can generate fine water droplets from a bulk water supply (under pressure or without pressure) and deliver those fine water droplets to the heating tube for heating and delivery to the skin surface of a user via the exit opening 48. Fine water droplets, for example, may have an average droplet size from 2 μm to 15 μm, from 4 μm to 10 μm, or from 6 μm to 8 μm, for example. Smaller droplets than those within these ranges may absorb into the skin to a higher degree that does not provide the user with a sufficiently wet experience, and furthermore, larger droplets than those within these ranges may provide a user experience where the skin becomes too wet, leading to significant water runoff from the skin and fast burn through of the water from the water supply tank. As the skin treatment device described herein is a handheld skin treatment device that contains its water reservoir within the handheld housing, a balance is also struck with providing adequate water for a more prolonged use without prematurely running out of water in the limited supply water storage tank. To illustrate, in examples of the present disclosure, the water supply tank can feed the atomizer at a rate of 70 mL of water per hour or greater, e.g., from 70 mL to 150 mL per hour, from 70 mL to 120 mL per hour, or from 80 mL to 100 mL per hour. Lower rates of water feed can provide some water to the skin surface, but at these higher rates of water delivery to the atomizer, a higher mist output with more coverage can be achieved. For example, the delivery of the heated water droplets at the cooled temperature can provide a delivery pattern having a skin surface contact area at least 10 times larger in area than the area of the exit opening, e.g., from 10 times to 25 times larger in area than the area of the exit opening. For example, if the cross-sectional area of the exit opening is from 2.5 cm² to 7.5 cm², then the coverage area when the exit opening is positioned from 5 cm to 20 cm from the skin surface may be from 25 cm² to 75 cm².

As water is delivered from the water supply tank 41 through the water delivery port 49 of the lower tank portion 45 to the heating tube 42, atomized water droplets pass through openings of an atomizer pressing cover 58. The atomizer pressing cover can be present to seal and connect the heater port to the heating tube. An absorption sheet 56 may likewise be present to provide absorption or water droplet runoff. The absorption sheet, along with the absorption swab 57, in some examples, may be collectively considered to be part of the absorption assembly mentioned previously. As water droplets coalesce and collect on the inside of the heating tube, water can run down and be collected at the absorption sheet to be wicked away via the absorption swab. Thus, in this example, the absorption sheet interfaces with the heating tube and the atomizer, and one side of the absorption sheet is extended. One end of the absorption swab is butted in an extended position with the absorption sheet. The absorption swab can be removed and dried or removed and replaced. When water absorbed by the absorption swab is wicked away from the atomizer and the absorption swab reaches a saturated state, the sprayer head (not shown, but shown in FIGS. 1A-2) can be opened for removal for wringing, drying, and/or replacement. Likewise, other arrangements of absorption assemblies can be used similarly to assist with keeping the atomizer drier for longer periods of time. Also shown is a retaining clip 59 to secure the assembly together. Other configurations and components may be used similarly.

Referring now to FIG. 6, a fixing adapter 50 can be included and installed as shown in FIGS. 1B and 4, for example, which is at a location at an upper end of the handheld housing about the heating tube (not shown in this FIG.). The middle of the fixing adapter is provided with a heating tube receiving channel 51 to receive the heating tube. The upper tank portion of the water supply tank (not shown) includes filling ports to fill water therein, and the fixing adapter includes a water channel 52 that corresponds with the filling ports so that the connection between the various components is compact and firm without the need to remove parts. Additionally, the fixing adapter is also provided with a swab receiving channel 53 to remove and replace the absorption swab. The fixing adapter also extends upward to form a stepped structure which may be fitted with an upper end of the handheld housing. The heating tube receiving channel and the water channel also both extend upward, while the swab receiving channel extends downward. By this design, the fixing adapter can seal the upper end of the handheld housing, and can also prevent the water channel, the heating tube, and the absorption swab from interfering with each other.

What is claimed is:

1. A handheld skin treatment device, comprising:
an atomizer to generate water droplets when fed from a water reservoir;
a water supply tank to contain the water reservoir and to feed water therefrom to interact with the atomizer and generate the water droplets; and
a heating tube to receive and heat the water droplets having an average temperature from 55° C. to 90° C. at an exit opening of the heating tube.

2. The handheld skin treatment device of claim 1, wherein the atomizer is an ultrasonic atomizer.

3. The handheld skin treatment device of claim 1, wherein the water droplets have an average droplet size from 2 μm to 15 μm.

4. The handheld skin treatment device of claim 1, wherein the water droplets have an average droplet size from 4 μm to 10 μm.

5. The handheld skin treatment device of claim 1, wherein the water droplets have an average droplet size from 6 μm to 8 μm.

6. The handheld skin treatment device of claim 1, wherein the water supply tank feeds the atomizer at a rate of greater than 70 mL of water per hour.

7. The handheld skin treatment device of claim 1, wherein the water supply tank feeds the atomizer at a rate of 80 mL to 100 ml of water per hour.

8. The handheld skin treatment device of claim 1, further comprising a battery that provides from 40 Watts to 100 Watts of power to the heating tube.

9. The handheld skin treatment device of claim 1, wherein the heating tube generates heated water droplets having an average temperature from 65° C. to 80° C. at an exit opening of the heating tube.

10. The handheld skin treatment device of claim 1, wherein the heated droplets are heated and of an average droplet size to have a temperature from 40° C. to 45° C. at a distance of from 5 cm to 10 cm from the exit opening of the heating tube.

11. The handheld skin treatment device of claim 1, wherein a portion of the water droplets as they are being passed through the heating tube collect on an interior surface of the heating tube, and wherein the interior surface is at a temperature where a portion of water droplets that collect on the interior surface become vaporized.

12. The handheld skin treatment device of claim 1, wherein the heating tube includes an interior surface of ceramic material.

13. The handheld skin treatment device of claim 1, wherein the heating tube has a 0.8 cm to 1.6 cm inner diameter and a 6 cm to 12 cm length.

14. The handheld skin treatment device of claim 1, wherein the heating tube passes through an opening defined by a wall of the water supply tank.

15. The handheld skin treatment device of claim 14, wherein the exit opening of the heating tube is located beyond where the heating tube passed through the opening defined by the wall of the water supply tank.

16. The handheld skin treatment device of claim 1, wherein an exterior surface of the heating tube is surrounded by a thermal insulator.

17. The handheld skin treatment device of claim 1, further comprising an absorption assembly to absorb water collected along an interior surface of the heating tube to slow the collection of water at the atomizer.

18. The handheld skin treatment device of claim 17, wherein the absorption assembly includes an absorption sheet with an opening therethrough that is aligned with the atomizer.

19. The handheld skin treatment device of claim 18, wherein the absorption assembly includes an absorption swab to wick water away from the absorption sheet to prolong the absorption volume provided by the absorption sheet.

20. The handheld skin treatment device of claim 1, further comprising a fixing adaptor to stabilize the heating tube and to provide an opening for filling the water supply tank.

21. A method of treating a skin surface with a handheld device, comprising:
atomizing water fed from a water reservoir held by a water supply tank within the handheld device to form water droplets;
passing the water droplets through a heating tube to form heated water droplets having an average temperature from 55° C. to 90° C. at an exit opening of the heating tube; and
directing the heated water droplets from the exit opening toward a skin surface, wherein the exit opening is positioned at from 5 cm to 15 cm from the skin surface to provide delivery of the heated water droplets at a cooled temperature from about 35° C. to about 45° C.

22. The method of claim 21, wherein the skin surface is a facial skin surface

[symbol]

23. The method of claim 21, wherein delivery of the heated water droplets at the cooled temperature provide a delivery pattern having a skin surface contact area at least 10 times larger than the exit opening.

24. The method of claim 21, wherein the heating tube is capable of delivering the heated water droplets from the exit opening while the heating tube is in any orientation.

25. The method of claim 21, wherein atomizing water is carried out using an ultrasonic atomizer.

26. The method of claim 21, wherein atomizing water results in water droplets having an average droplet size from 2 μm to 15 μm.

27. The method of claim 21, further comprising providing battery power resulting in energy input to the heating tube from 40 Watts to 100 Watts.

28. The method of claim 21, further comprising reducing a volume of the water droplets that collect on an atomizer used for atomizing the water by:
vaporizing water droplets collected on an interior surface of the heating tube,
absorbing runoff of water droplets along the interior surface of the heating at an absorption assembly, or
both.
